(19) United States
(12) Patent Application Publication
(10) Pub. No.: US 2012/0266699 A1
(43) Pub. Date: Oct. 25, 2012

(54) LUBRICANT FOR WEAR TESTING OF JOINT REPLACEMENTS AND ASSOCIATED MATERIALS

(76) Inventors: Jan-M. Brandt, Winnipeg (CA); Kory D.J. Charron, St. Thomas (CA); Lin Zhao, London (CA); John B. Medley, Fergus (CA); Steven J. MacDonald, London (CA)

(21) Appl. No.: 13/274,088

(22) Filed: Oct. 14, 2011

Related U.S. Application Data
(63) Continuation of application No. 12/069,280, filed on Feb. 7, 2008, now abandoned.
(60) Provisional application No. 60/899,894, filed on Feb. 7, 2007.
(60) Provisional application No. 61/006,102, filed on Dec. 19, 2007.

(51) Int. Cl. C10M 141/10; G01N 33/00

(52) U.S. Cl. 73/865.6; 508/162

(57) ABSTRACT

The present invention relates to an artificial synovial fluid composition that mimics the tribological and biochemical properties of human, osteoarthritic synovial fluid for in vitro testing of implantable materials. The artificial synovial fluid comprises new-born calf serum or alpha-calf serum (iron-free or iron-supplemented) that contains a specific total protein concentration, specific protein constituent fractions (albumin, α-1 globulin, α-2 globulin, β-globulin, and γ-globulin), specific trace element concentrations (Ca, Mg, inorganic P, and Fe), and a specific low-molecular weight peptide concentration important for in vitro testing of implantable materials. In addition, the artificial synovial fluid contains a hyaluronic acid concentration and level of osmolality (preferably using phosphate buffered-saline solution) to mimic the thermal stability (transition mid-point temperature, enthalpy, and entropy) and pH level as measured clinically.

### RELATED APPLICATIONS

This application is a continuation of U.S. patent application Ser. No. 12/069,280 filed Feb. 7, 2008, which claims priority from U.S. Provisional Application No. 60/899,894 filed Feb. 7, 2007 and U.S. Provisional Application No. 61/006,102 filed Dec. 19, 2007, the entire disclosures of which are incorporated herein by reference.

### FIELD OF THE INVENTION

The present invention relates to an artificial synovial fluid composition that mimics the tribological properties of human, osteoarthritic synovial fluid (SF) for the main purpose of in vitro wear testing. More specifically, the invention relates to a composition having specified total serum protein concentration, specific fractions of serum protein constituents, peptide concentration, level of osmolality (an indicator of the ionic strength of a solution), hyaluronic acid (HA) concentration and antimicrobial agents to simulate synovial fluid wear properties in a tribological analysis of an artificial joint in a sterile environment.

### BACKGROUND OF THE INVENTION

Artificial joints are typically subjected to simulator wear testing to evaluate the influence of materials and designs on wear. One of the main goals of simulator wear tests is to gain some insight into the possible in vivo wear performance of the implant (Walker et al. (1997) J. Biomech. 30(1):83-89; Walker et al. (2000) Clin. Orthop. 472:290-301; Burgess et al. (1997) Proc. Inst. Mech. Eng. J. Eng. Med. 211(1):37-47; and Barnett et al. (2002) Proc. Inst. Mech. Eng. [H] 216(1):51-61) and, thus, simulator wear testing conditions should closely mimic those of the in vivo situation in order to best estimate the wear rate of the artificial joint. Wear particles in vivo can initiate periprosthetic osteolysis, a serious complication in joint replacement arthroplasty that makes expensive revision surgery necessary. Features of a wear system in joint replacements include the contact geometry, bearing material properties, load, motion, surface roughness, test temperature and lubricant.

The use of a bovine serum lubricant in wear simulator testing of polyethylene pins on cobalt alloy plates was shown by McKellop et al. (1978, J. Biomed. Mater. Res. 12(6):895-927) to cause surface damage similar to that observed in retrieved total hip replacements having this same material combination. Similar results were not obtained when either distilled water (DW) or saline solution was used as the lubricant. This research suggested that serum proteins played an important role in a boundary lubrication process that influenced the wear of the polyethylene (PE) bearing surface (Liao et al. (1999) J. Biomed. Mater. Res. 48(4):465-473; Wang et al. (1999) Trans. Orthop Res. Soc. 24; Good et al. (2000) 71(4):365-369; Clarke et al. (2001) Wear 250(1-12):188-198; and Kaddick and Wimmer (2001) Proc. Inst. Mech. Eng. [H] 215(5):429-442). Most recent protocols for joint simulator wear testing use lubricating fluids that consist of DW or saline solutions with bovine serum and a small quantity of an anti-microbial agent to limit microbial growth that would not occur in vivo (Bigsby et al. (1997) Proc. Inst. Mech. Eng. Pt. H 211:265-269; Clarke et al. (1995) as presented at Trans. Combined ORS Meeting, San Diego, Orthop. Res. Soc. p. 204; and Wang et al. (1995) Biomaterials 17:865-871).

Lubricants that simulate human SF are likely to be important in the realistic evaluation of the wear of joint replacement implants. Unfortunately, they vary between wear test laboratories. Bovine, newborn calf and alpha calf serum, have all been part of the lubricants used in wear testing and each has different amounts and types of proteins (Clarke et al. (2001) Wear 250(1-12):188-198; Kaddick and Wimmer (2001) Proc. Inst. Mech. Eng. [H] 215(5):429-442; McEwen et al. (2005) J. Biomech. 38(2):357-365). The International Standards Organization (ISO) has devised standards for knee wear simulator testing that are currently followed by many wear test laboratories. In these standards, the lubricant is calf serum of some type diluted with de-ionized water to a total protein concentration of 17 g/L (ISO-14243-3 (2004) International Organization for Standardization, London: Implants for Surgery: Wear of Total Knee Joint Prostheses, Part 3: Displacement Control and Corresponding Environmental Conditions for Test; ISO-14243-2 (2002) International Organization for Standardization, London: Implants for Surgery: Wear of Total Knee Joint Prostheses, Part 1: Control and Corresponding Environmental Conditions for Test). By not specifying the calf serum type, the standards avoid giving specific fractions of the protein constituents and each of these serum protein constituents is likely to have a unique effect on wear. The ISO also specifies the use of sodium azide (SA) as an anti-microbial agent.

Anti-microbial agents such as SA, antibiotics and antimycotics have been added in various combinations by some laboratories to the serum-based lubricants used in wear testing in order to suppress or prevent microbial growth (McEwen et al. (2005) J. Biomech. 38(2):357-365; Chan et al. (1999) Trans Ortho Res. Soc. 45:310; and Schwenke et al. (2005) Proc. Inst. Mech. Eng. [H] 219(6):457-464). Although Bell et al. (2000, Proc. Inst. Mech. Eng. [H] 214(5):513-518) showed that SA was not effective after 28 h of wear testing in a pin-on-disc arrangement, the influence of microbial growth on simulator wear testing has not been established.

It has been further demonstrated that the various types of calf serum used in the lubricants for simulator wear testing have highly variable chemical compositions with respect to serum protein concentration and ion content (Polineni et al. (1997) Trans 23^{rd} Annual Meeting of the Society for Biomaterials, New Orleans, La., p. 154; Wang et al. (1999) 45^{th} ORS, Anaheim, Calif., p. 52; and Wang et al. (1998) Trans 24^{th} Annual Meeting of the Society for Biomaterials, p. 218). Wang et al. (2004, J. Biomed. Mater. Res. 68B(1):45-52) showed that the albumin-to-total globulin ratio of serum and de-ionized water influenced the wear rate. At a ratio of 4:1 (when using alpha calf serum), the PE wear rate was approximately 64% higher than the ratio of 2:1 when using bovine calf serum.

The wear process may damage the proteins and, thus, initiate unfolding, precipitation and shear of the proteins (FIG. 1). Protein unfolding may be promoted by the effects of load and motion during the wear test that generates heat at the CoCr-PE interface.

The proteins themselves consist of amino acids linked together by peptide bonds; connected links of amino acids are referred to as polymeric peptides or simply polypeptides (Sugio et al. (1999) Protein Eng. 12(6):439-446). Such polymeric peptides are known to adsorb onto hydrophobic surfaces via a thermodynamically driven process (Norde et al. (1991) J. Biomater. Sci. Polym. Ed. 2(3):183-202; Norde and Lyklema (1989) Colloids and Surfaces 38:1-13; Hlady and Buijs (1996) Curr. Opin. Biotechnol 7(1):72-7; Malmsten (1998) J. Colloid Interface Sci. 207:186-199; Haynes and Norde (1995) J. Colloid Interface Sci. 169(2):313-328; Serro et al. (2006) J. Biomed. Mater. Res. A 78(3):581-589). Tsarkova et al., 2004 (Colloid J. 66(1):84-94) and Tsarkova et al., 2007 (Macromolecules 40(7):2539-2547) showed that the polymeric chains adsorbed to each surface were highly interactive, further demonstrating that the head groups of the polymeric chains were entangled with each other. Upon sliding, the entangled polymers were shown to stretch, creating shear force and increasing the friction coefficient. Under such circumstances, it was postulated that mechanical shear could cause the larger polypeptides to break into shorter peptide chains (Yuh-Fun (1996) Biotechnol. Bioengineering 51(4):458-465).

Hyaluronic acid (HA) is a polymeric disaccharide forming a major natural constituent of SF. HA is largely responsible for the viscosity of SF. Oates et al. suggested that HA, in presence of albumin, generates a strong protein-HA network with rheopectic behaviour at low shear rates (Oater et al. (2006) J. R. Soc. Interface 3(6):167-174). Such rheopectic behaviour suggested that the viscosity of SF may increase with increased duration of shear. Wang et al. added 0.34 g/L of HA to alpha calf serum diluted with deionized water to a total protein concentration of 35 g/L (Wang et al. (J. Biomed. Mater. Res. 68B(1):45-52). It was further demonstrated that the addition of HA to diluted calf serum had no effect on the PE wear rate after a 1 million cycle (Mc) hip simulator wear test. Desjardins et al. added 1.5 g/L HA, having a molecular weight of 2.3 MDa, to diluted bovine calf serum having a total protein concentration of 35 g/L and conducted a 5 Mc wear testing using a force-controlled knee simulator (Desjardins et al. (2006) Proc. Inst. Mech. Eng. [H] 220(5):609-623). In this study, a 6.8-fold increase in PE wear rate was reported in situations of a lubricant containing HA as well as SA as the anti-microbial agent.

Heat may be generated by contacting asperities of two surfaces in relative motions (Kuhlmann-Wilsdorf (1985) Wear 105(3):187-198). In particular, heat accumulation at the interface of the artificial joint articulation may facilitate the protein degradation and increase the PE wear rate (Liao et al. (2003) Biomaterials 24(18):3047-3059). Recently, Heuberger et al. (Heuberger et al. (2005) Biomaterials 26(10):1165-1173) and Fang et al. (Fang et al. (2007) Appl. Surface Sci. 253(16):6896-6904) showed that a lubricant containing thermally degraded albumin proteins caused a higher friction than a lubricant contain native albumin proteins. The thermal stability of the protein-rich lubricant depends on the ionic strength and can be determined by assessing the calorimetric characteristics of the lubricant using Differential Scanning calorimetry (Giancola et al. (1997) Int. J. Biol. Macromol. 20(3):193-204). The osmolality of the solution is the concentration of osmotically active particles (for example NaCl ions) in a solution (Madigan and Martinko (2006), In: Biology of Microorganisms, 11^{th} Edition, 274:693). The osmolality of SF depends on the activity of the patient (Baumgarten et al. (1985) J. Bone Joint Surg. Am. 67(9):1336-1339). In SI units, patients at rest typically have an osmolality of 404±57 mmol/kg while patients after exercise typically have an osmolality of 303.3±1.5 mmol/kg.

There remains a need to identify improved compositions that mimic in vivo synovial fluid for wear simulator testing of artificial joints. Close approximation of wear testing conditions to the in vivo situation is critical for accurate evaluation of artificial joint wear.

### SUMMARY OF INVENTION

In accordance with the purpose(s) of this invention, as embodied and broadly described herein, this invention relates to an artificial synovial fluid. In one embodiment, the present invention provides an artificial synovial fluid composition comprising: a) a mammalian serum having i) a total protein concentration of from about 10 to about 45 g/L; ii) an albumin fraction of from about 55% to about 80% w/w %; iii) an α-1 globulin fraction of from about 3% to about 6% w/w %; iv) an α-2 globulin fraction of from about 5% to about 10% w/w %; v) a β-globulin fraction of from about 3% to about 20% w/w %; vi) a γ-globulin fraction of from about 5% to about 20% w/w %; vii) a calcium (Ca) concentration of from about 0.1 mmol/L to about 3 mmol/L; viii) a magnesium (Mg) concentration of from about 0.05 mmol/L to about 0.8 mmol/L; ix) an inorganic phosphate (P) concentration of from about 0.1 mmol/L to about 1.5 mmol/L; x) an iron (Fe) concentration of from about 0.001 mmol/L to about 0.1 mmol/L; xi) a peptide concentration at a 2,000 Da molecular weight cut-off of from about 0.005 g/L to about 10 g/L; xii) a maximal transition midpoint temperature, T_{m-cp-max}, of from about 330K to about 350K at a concentration molality of from about 0.005 to about 0.15 mmol/L measured at a scan rate of from about 20 K/hour to about 100K/hour; xiii) a total enthalpy change, ΔH, of from about 650 kJ mol⁻¹ to about 1200 kJ mol⁻¹ at a set concentration molality of 0.05 mmol/1 measured at a scan rate of 60 K/hour; xiv) a total entropy change, ΔS, of from about 1.5 kJ mol⁻¹ K⁻¹ to about 4 kJ mol⁻¹ H⁻¹ at a concentration molality of from about 0.005 mmol/l to about 0.15 mmol/l and a scan rate from about 20K/hour to 100K/hour; wherein the ratio of albumin to total globulin (α-1 globulin+α-2 globulin+β-globulin+γ-globulin fractions) in solution is from about 1.5 to about 3; b) a buffer for maintaining a solution osmolality of from about 200 mmol/kg to about 400 mmol/kg; c) hyaluronic acid (HA) at a concentration of from about 0.1 g/L to about 6 g/L; d) hyaluronic acid (HA) with a molecular weight of from 1 MDa to 4 MDa; and e) a pH level of about 7 to about 8.

### BRIEF DESCRIPTION OF FIGURES

The following drawings form part of the present specification and are included to further demonstrate certain embodiments. These embodiments may be better understood by reference to one or more of these figures in combination with the detailed description of specific embodiments presented herein.

FIG. 1 illustrates the potential effects of the wear process on the native, folded serum protein structure, and indicates that wear may lead to protein damage and cause protein unfolding, protein precipitation and protein shear according to an embodiment of the present invention.

FIG. 2 illustrates the lubricant protein precipitation and centrifugation according to an embodiment of the present invention. Fresh, unworn lubricant is referred to as “starting material” (SM). After the wear test, the worn lubricant contains suspended protein precipitates. Centrifugation of the worn lubricant separates the protein precipitates from the remaining lubricant in the pellet (SUP=supernatant).

FIG. 3 illustrates the total protein concentration in human synovial fluid (SF) samples obtained from twenty patients with osteoarthritis as their primary diagnosis according to an embodiment of the present invention.

FIG. 4 illustrates the fraction (%) of proteins including albumin, α-1-globulin, α-2-globulin, β-globulin and γ-globulin in SF as well as in various types of serum lubricants (BCS: bovine calf serum (Lot# AQC23290); NCS: newborn calf serum (Lot# APE21200); ACS: alpha-calf serum (Lot# AQE23894); all from HyClone Inc., Logan, Utah) according to an embodiment of the present invention.

FIG. 5 illustrates the osmolality (mmol/kg) of synovial fluid (SF), BCS, NCS, ACS and ACS-I diluted with either de-ionized water (DW) or phosphate buffered saline (PBS) (VWR; Cat. #72060-034) to a final protein concentration of 17 g/L according to an embodiment of the present invention. The osmolality of PBS was 286±0.57 mmol/kg, approximately 6-fold higher than that of DW having an osmolality of 46±2.08 mmol/kg) according to an embodiment of the present invention.

FIG. 6 illustrates the concentration (mmol/kg) of trace elements calcium (Ca), magnesium (Mg), inorganic phosphate (P) and iron (Fe) in each of SF, undiluted BCS, undiluted NCS, undiluted ACS and undiluted ACS-I according to an embodiment of the present invention.

FIG. 7 illustrates the AMK total knee system (DePuy Orthopaedics, Inc., Warsaw, Ind.). The patella was not used in wear tests.

FIG. 8 illustrates the wear rates for serum lubricants in the absence of either one or both of HA and PBS according to an embodiment of the present invention. BCS, NCS, and ACS were diluted with DW and SA according to an embodiment of the present invention.

FIG. 9 illustrates a summary of the protein constituent degradation for the serum lubricants according to an embodiment of the present invention.

FIG. 10 illustrates the correlation between wear rate and initial combined albumin+a-globulin fraction for various lubricants according to an embodiment of the present invention. Each data point represents the value obtained from a single wear test according to an embodiment of the present invention.

FIG. 11 illustrates the correlation between protein degradation during the wear testing and initial combined β-globulin+γ-globulin fraction according to an embodiment of the present invention. Four protein degradation values were determined for each wear station such that each data point represents a single value according to an embodiment of the present invention.

FIG. 12 illustrates an isolated Enterobacter Cloacae JK-1 bacterium (E. cloacae JK-1) present in the BCS lubricant after a test interval of 0.5 Mc (shown in a micrograph).

FIG. 13 illustrates the number of colony-forming units per mL serum (CFU/mL) for the BCS, NCS and ACS lubricants through a test interval from 0-0.5 Mc according to an embodiment of the present invention.

FIG. 14 illustrates the protein degradation profile for lubricants BCS+DW, NCS+DW, and ACS+DW when either SA or AA was used as the microbial inhibitor according to an embodiment of the present invention. It appeared that the protein degradation was not different for NCS+DW and ACS+DW.

FIG. 15 illustrates the peptide concentration of BCS+DW, NCS+DW and ACS+DW after 0.5 Mc in the presence of SA or AA as the microbial inhibitor according to an embodiment of the present invention. The SUP's of each lubricant were ultra-filtrated using a membrane with a molecular weight cut-off of 2,000 Da according to an embodiment of the present invention. It appeared there was a similarity in the relative increase in peptide concentration for BCS+DW, NCS+DW, and ACS+DW in the presence of AA compared to SA.

FIG. 16 illustrates a schematic representation of a thermogram according to an embodiment of the present invention.

FIG. 17 illustrates the thermogram of ACS-I+DW+AA (triplicate measurements) according to an embodiment of the present invention.

FIG. 18 illustrates the thermogram of ACS-I+PBS+AA (triplicate measurements) according to an embodiment of the present invention.

FIG. 19 illustrates the thermogram of ACS-I+PBS+AA+HA (triplicate measurements) according to an embodiment of the present invention.

FIG. 20 illustrates the thermogram of SF 7 (triplicate measurements) according to an embodiment of the present invention.

FIG. 21 illustrates the thermogram of SF 8 (triplicate measurements) according to an embodiment of the present invention.

FIG. 22 illustrates the thermogram of SF 18 (triplicate measurements) according to an embodiment of the present invention.

FIG. 23 illustrates the change in enthalpy, ΔH, for the DW lubricant, the PBS lubricant, the HA lubricant and SF (SF 7, SF 8, SF 18) according to an embodiment of the present invention. It appeared there was a similarity in magnitude between the HA lubricant and SF.

FIG. 24 illustrates the change in entropy, ΔS, for the DW lubricant, the PBS lubricant, the HA lubricant, and SF (SF 7, SF 8, SF 18) according to an embodiment of the present invention. It appeared there was a similarity between the HA lubricant and SF.

FIG. 25 illustrates the wear rates for serum lubricants diluted in PBS with or without HA (L implants) according to an embodiment of the present invention.

FIG. 26 illustrates the protein constituent concentrations for the SFₘᵢₓ starting material (SM; protein concentration=30.90±1.24 g/l) and the SFₘᵢₓ supernatant (SUP; 25.98±1.31 g/l) after a test interval of 5,000 cycles according to an embodiment of the present invention. It appeared all protein sub-constituents were affected by the wear process.

FIG. 27 illustrates the wear rates for serum lubricants diluted with PBS or DW (R implants) according to an embodiment of the present invention.

FIG. 28 illustrates the peptide concentration for ACS+DW+SA, ACS+DW+AA, and ACS-I+DW+AA (MWCO=2,000 Da) according to an embodiment of the present invention. It appeared there was an 11-fold increase in peptide concentration for ACS+DW when SA was replaced with AA. The peptide concentration did not appear significantly different between ACS+DW+AA and ACS-I+DW+AA (p=0.170, ANOVA and Tamhane), despite the higher Fe concentration in the latter lubricant.

FIG. 29 illustrates the wear rate for ACS+DW+SA and ACS-I+DW+AA according to an embodiment of the present invention. It appeared there was a 4-fold decrease in wear rate when SA was replaced with AA, assuming that the effects of increased Fe in ACS-I on PE wear were negligible.

FIG. 30 illustrates the schematic of CoCr-PE asperity contact with a low peptide concentration when SA was used as the microbial inhibitor (ACS+DW+SA) according to an embodiment of the present invention.

FIG. 31 illustrates a schematic of CoCr-PE asperity contact with a high peptide concentration when AA was used as the microbial inhibitor (ACS-I+DW+AA) according to an embodiment of the present invention. There appeared to be a preferred adsorption of low-molecular weight peptides onto the bearing surfaces, forming a low-wear peptide layer (conditioning film) that significantly reduced adhesive/abrasive wear.

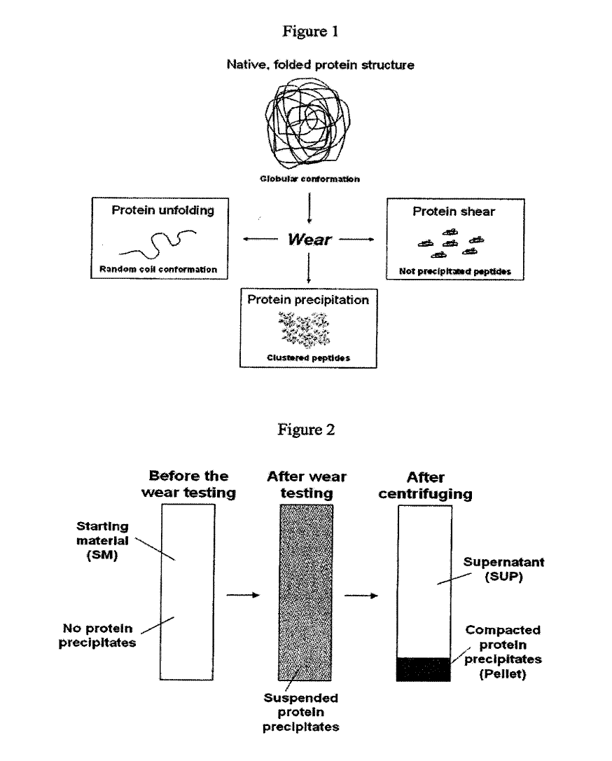

### DETAILED DESCRIPTION

In the following description, numerous specific details are set forth to provide a thorough understanding of the invention.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present invention and/or methods are disclosed and described. it is to be understood that this invention is not limited to specific reagents or synthetic procedures, as such may, of course, vary, unless it is otherwise indicated. It is also to be understood that the terminology used herein is for the purpose of describing particular embodiments only and is not intended to be limiting.

The present artificial synovial fluid composition of the present invention mimics in vivo synovial fluid for use in wear simulator testing of artificial joint replacements. The artificial synovial fluid may further comprise a microbial inhibitor selected from the group consisting of a β-lactam antibiotic; a β-lactamase-sensitive penicillin; a β-lactamase inhibitor; a combination β-lactam antibiotic and beta-lactamase inhibitor; an aminoglycoside; and a bacteriostatic, wherein the microbial inhibitor is present at a concentration of from about 1% to about 15% total volume.

An antifungal agent may also be present, which may be selected from the group comprising Amphotericin B, Fungilin, Abelcet, AmBisome, Fungisome, Amphocil, and Amphotec.

The β-lactam antibiotic may be selected from the group comprising Ampicillin, Pivampicillin, Carbenicillin, Amoxicillin, Carindacillin, Bacampicillin, Epicillin, Pivmecillinam, Azlocillin, Mezlocillin, Mecillinam, Piperacillin, Ticarcillin, Metampicillin, Talampicillin, Sulbenicillin, Temocillin, and Hetacillin.

The β-lactamase sensitive penicillin may be selected from the group comprising Benzylpenicillin, Phenoxymethylpenicillin, Propicillin, Azidocillin, Pheneticillin, Penamecillin, Clometocillin, Benzathine benzylpenicillin, Procaine benzylpenicillin, Benzathine phenoxymethylpenicillin, Dicloxacillin, Cloxacillin, Methicillin, Oxacillin, and Flucloxacillin.

The β-lactamase inhibitor may be selected from the group comprising Sulbactam and Tazobactam.

The combination penicillin and beta-lactamase inhibitor may be selected from the group comprising Ampicillin and an enzyme inhibitor; Amoxicillin and an enzyme inhibitor, Ticarcillin and enzyme inhibitor, Sultamicillin Piperacillin and enzyme inhibitor, combinations of penicillins) including beta-lactamase inhibitors (Ampicillin and enzyme inhibitor, Amoxicillin and enzyme inhibitor, Ticarcillin and enzyme inhibitor, Sultamicillin Piperacillin and enzyme inhibitor, combinations of penicillins).

The aminoglycoside may be selected from the group comprising Streptomycin, Streptoduocin, Tobramycin, Gentamicin, Kanamycin, Neomycin, Amikacin, Netilmicin, Sisomicin, Dibekacin, Ribostamycin, and Isepamicin.

The bacteriostatic may be selected from the group comprising Tetracycline, Sulphonamide, Spectinomycin, Trimethoprim, Chloramphenicol, Macrolide and Lincosamide.

Any anti-infectives for systemic listed in the Anatomical Therapeutic Chemical Classification System JO1 (ATC J01) may be used in the synovial fluid.

The mammalian serum may be a bovine serum selected from the group comprising alpha calf serum (ACS), ACS-I, bovine calf serum (BCS) and newborn calf serum (NCS).

The buffer may comprise sodium chloride, sodium phosphate and potassium phosphate. The buffer may be selected from the group comprising PBS.

The artificial synovial fluid composition may have a total protein concentration of from about 22 g/L to 44 g/L. The albumin fraction may be from about 60% to about 75% w/w %. The α-1 globulin concentration may be from about 3.3% to about 5.2% w/w %. The α-2 globulin concentration is from about 5.8% to about 9.7% w/w %. The β-globulin concentration may be from about 3.4% to about 17.7% w/w %. The γ-globulin concentration may be from about 4.8% to about 12.9% w/w %. The hyaluronic acid (HA) concentration may be from about 1 g/L to about 2 g/L. The molecular weight of hyaluronic acid (HA) may be from about 1.5 MDa to about 3.5 MDa.

The calcium (Ca) concentration may be from about 0.32 mmol/L to about 2.2 mmol/L. The magnesium (Mg) concentration may be from about 0.04 mmol/L to about 0.7 mmol/L. The inorganic phosphate (P) concentration may be from about 0.2 mmol/L to about 1.1 mmol/L. The iron (Fe) concentration may be from about 0.004 mmol/L to about 0.05 mmol/L. Finally the peptide concentration may be at a 2,000 Da molecular weight cut-off of from about 0.05 g/L to about 2 g/L.

The artificial synovial fluid composition may have a maximal transition midpoint temperature, T_{m-cp-max}, of from about 336 K to about 338 K at a concentration molality of from about 0.005 to about 0.15 mmol/l and a scan rate from about 20K/hour to 100K/hour. The total enthalpy change, ΔH, may be from about 700 kJ mol⁻¹ to about 1050 kJ mol⁻¹ at a set concentration molality of 0.05 mmol/1 measured at a scan rate of 60 K/hour.

The artificial synovial fluid composition may have a total entropy change, ΔS, is from about 2 kJ mol⁻¹ K⁻¹ to about 3.2 kJ mol⁻¹ K⁻¹ at a set concentration molality of 0.05 mmol/1 measured at a scan rate of 60 K/hour. The ratio of albumin to total globulin (α-1 globulin+α-2 globulin+β-globulin+-globulin fractions) in solution may be from about 1.8 to about 2.3.

The artificial fluid composition may be used for wear testing artificial joint implants in in vitro simulators. In particular, the artificial joint implant may be selected from the group consisting of a knee joint implant, a hip joint implant, a shoulder implant, a spinal implant, and any other joint replacement implant or implant surfaces that are under relative motion during their implantation in a mammalian body. Also described is a method of joint implant wear simulator testing in vitro comprising subjecting the joint implant to wear simulator testing in the presence of an artificial synovial fluid composition as described above.

As used herein, the term “serum” refers to the clear, protein-rich portion of any body fluid. In particular, serum refers to “blood serum”, the cell-free, protein-rich, fluid portion of the blood. Serum can refer to human serum or other mammalian serum. Constituent serum proteins include albumin, α-1-globulin, α-2-globulin, β-globulin and γ-globulin.

As used herein, the term “peptide” refers to a compound of at least two amino acids. The peptide has a molecular weight of no more than 2000 Da.

As herein used, the term “knee simulator” refers to a six station AMTI knee simulator (KS3-6-1000, Serial #120219, AMTI, Waltham, Mass.). This simulator was used to specifically test total knee replacement implants under physiological loading and motions. The simulator consisted of a left (L) bank and a right (R) bank with separate load and motion actuators. The implants used on the L bank and the R bank were referred to as the “L implants” and “R implants”, respectively. Each bank had three dynamic wear stations and two load-soak (LS) stations.

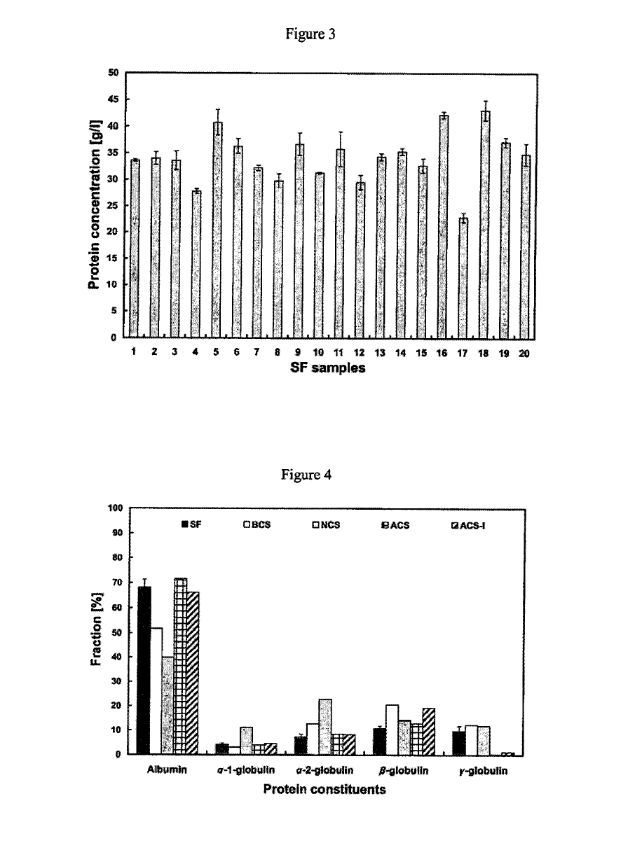

The lubricants used in the wear simulations were damaged during the wear process. Such damage led to protein degradation that had to be characterized to be able to explain the overall acting wear mechanism. At the beginning of the wear tests the fresh lubricant used as the “starting material (SM)” was often translucent and yellow. As the wear test progressed, the lubricants lost their translucent feature and became opaque. Such altered visual appearance suggested that some protein constituents were damaged and had precipitated out of solution but remained suspended in the lubricant (FIG. 2). To separate the damaged proteins from the unworn proteins the lubricant samples had to be centrifuged. This caused the suspended proteins to become compacted on the bottom of the tubes which resulted in a pellet. The fluid on top of the pellet was called the supernatant (SUP) and was free of any precipitates.

As used herein, the term “antibiotic” refers to a microbial inhibitor. Common antibiotics include antibiotic-antimycotics (AA) which target a broad spectrum of bacteria, fungi and yeast. Antibiotics for use with the invention include β-lactams (e.g. penicillin, carbenicillin), aminoblycosides (e.g. streptomycin, tetracycline), and bacteriostatics (e.g. chloramphenicol). In particular, the AA contains 10,000 units of penicillin, 10,000 μg streptomycin, 25 μg amphotericin B/mL, and includes streptomyucin sulphate. In particular, the antimycotic is amphotericin B (Fungizone®).

As used herein, the term “buffer” refers to phosphate-buffered saline solution. The buffer may include both HEPES and TRIS can be used to maintain pH 7.0-7.4. HEPES (4-(2-hydroxyethyl)-1-piperazineethanesulfonicacid) couples with NaOH and TRIS (trishydroxymethylaminomethane or the IUPAC name 2-amino-2-hydroxymethyl-1,3-propanediol) with HCl at a concentration of approximately 20-50 mM.

### EXAMPLES

The following examples are included to demonstrate particular embodiments of the invention. It should be appreciated by those of skill in the art that the techniques disclosed in the examples which follow represent techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments which are disclosed and still obtain a like or similar result without departing from the spirit and scope of the invention.

### Example 1

### Serum Protein Concentration and Protein Constituent Fractions Vary Depending on the Lubricant

The ratio of albumin/globulin in serum lubricant at the start of a wear test has been used as an indicator of wear in several hip simulator studies (Wang et al (2004) J. Biomed. Mater. Res. 68B(1):45-52), however, determination of clinically relevant fractions of specific serum proteins, including α-1, α-2, β, and γ-globulins, in wear simulator testing has not been evaluated, nor has the relevance of these protein fractions in wear simulator testing.

Twenty patients were selected to participate in the present study: ten male and ten female patients, with a mean age of 64.7 years (range, 60-70 years), undergoing surgery for primary total knee arthroplasy (Table 1). Patients who had previously received any knee injections for pain relief (i.e. Cortisone (a steroid hormone) or Synvisc® HYLAN G-F 20 (Genzyme, Cambridge, Mass.)), or patients with rheumatoid arthritis (RA) or other inflammatory arthritis, were eliminated from the study. Also, patients were excluded who were undergoing revision surgery or who had received a high tibial osteotomy. Ten patients underwent right knee surgeries and ten underwent left knee surgeries. Based on the pre-admission notes, the patient-specific body-mass-index (BMI) measured on average 31 (range, 26-41 BMI). SF was aspirated from twenty patients just prior to surgical replacement of the knee with a primary total knee replacement (Ethical Board Review Number 12536E, University of Western Ontario, ON, Canada), by four independent physicians. Sterile techniques, as per hospital protocol, were maintained to protect the patient. The amount of SF aspirated varied (range, 1-8 ml), with a mean of 4 ml.

TABLE 1
Nomenclature and patient characteristics of SF samples.

| SF | Body-mass index | | | |
|---|---|---|---|---|
| identification | [BMI] | Gender | Knee | Age |
| SF1 | 41 | F | Left | 61 |
| SF2 | 33 | F | Left | 64 |
| SF3 | 31 | M | Left | 62 |
| SF4 | 31 | F | Right | 68 |
| SF5 | 24 | F | Right | 63 |
| SF6 | 40 | F | Left | 69 |
| SF7 | 26 | M | Left | 65 |
| SF8 | 30 | M | Left | 70 |
| SF9 | 27 | F | Right | 66 |
| SF10 | 29 | M | Right | 70 |
| SF11 | 32 | F | Right | 64 |
| SF12 | 38 | F | Left | 62 |
| SF13 | 31 | F | Right | 65 |
| SF14 | 26 | M | Left | 63 |
| SF15 | 38 | F | Right | 61 |
| SF16 | 27 | M | Right | 60 |
| SF17 | 26 | M | Left | 64 |
| SF18 | 31 | M | Left | 60 |
| SF19 | 30 | M | Right | 66 |
| SF20 | 36 | M | Right | 70 |

The serum protein fractions of four calf sera (bovine calf serum (BCS), newborn calf serum (NCS), alpha-calf serum (ACS) and iron-supplemented alpha-calf serum (ACS-I)) were determined (all obtained from Hyclone Inc. (Logan, Utah)). The protein compositions of the various sera are provided in Table 2. Five hundred millilitres of calf sera were obtained and stored at −20° C. until use. Individual certificates of analysis were obtained from the serum manufacturer for each batch of serum used to provide data on the individual protein and inorganic component concentrations. The total protein concentration and individual protein fractions varied to some extent across the different sera. The albumin fraction of ACS was consistently found to be highest and most similar to that of ACS-I.

TABLE 2
Characteristics of four calf sera. Note the difference in constituents.

| Specifications | BCS | NCS | ACS | ACS-I |
|---|---|---|---|---|
| Lot # | AQC23290 | APE21200 | APL22372 | AQE23894 |
| Albumin fraction | 51.7 | 40 | 71.7 | 66.3 |
| [%] | | | | |
| α-1-globulin | 3 | 11.2 | 3.8 | 4.5 |
| fraction [%] | | | | |
| α-2-globulin | 12.7 | 22.8 | 8.4 | 8.2 |
| fraction [%] | | | | |
| β-globulin fraction | 20.4 | 14.2 | 15.8 | 19.2 |
| [%] | | | | |
| γ-globulin fraction | 12.2 | 11.8 | 0.3 | 1.1 |
| [%] | | | | |
| Total protein [g/l] | 69 | 52 | 42 | 41 |
| Fe [mmol/L] | 0.0065 | 0.0157 | 0.0085 | 0.0877 |
| Ca [mmol/L] | 2.78 | 2.70 | <0.5 | <0.5 |
| Inorganic P | 3.16 | 2.82 | <0.2 | <0.2 |
| [mmol/L] | | | | |
| Mg [mmol/L] | 1.02 | 1.06 | 0.06 | 0.08 |
| pH | 7.29 | 7.49 | 7.65 | 7.71 |
| Osmolality | 301 | 290 | 291 | 283 |
| [mmol/kg] | | | | |

Total protein concentrations of the patient SF samples (FIG. 3) and various calf sera (Table 2) were determined using a bicinchonic acid protein assay kit (BCA™ protein assay kit, Cat. # P123227, Pierce Chemicals, Rockford, Ill.). In this assay, a biuret reagent (copper sulphate, a strong base) undergoes a chemical reaction, as the reaction between the peptide bonds of a protein and the Cu atoms results in the formation of a coloured complex. Protein concentrations were calculated as g/L.

Proteins extracted from SF and sera samples were subjected to electrophoresis to identify the proteins present in the samples based on protein net charge, the viscosity of the protein mixture, and protein size. The proteins with a smaller radius were expected to migrate more rapidly than proteins with larger radii. The apparatus used for the electrophoresis (Sebia Hydrasys®, Sebia, Norcross, Ga.) permitted the electrophoresis of 30 individual samples on a single alkaline-buffered (pH=8.5) agar-based gel (Hydragel 30 β1-β2, Sebia, Norcross, Ga.). The gel separated five major protein fractions: albumin, α-1 globulin, α-2 globulin, β-globulin and γ-globulin. The SF was pre-treated with hyaluronidase, an enzyme that catalyzes the breakdown of HA and increases the permeability of the protein constituents into the gel, to reduce the viscosity of the lubricant. Hyaluronidase powder (Hyalurono-glucosaminidase, H 3506, Sigma-Aldrich, St.Louis, Mo.) was diluted with PBS to a 71 g/l solution and was then added to 100 μl of synovial fluid in a ratio of 1:20. Hyaluronidase-treated samples were electrophoresed separately and served as controls.

The fractions of serum proteins albumin and α-1, α-2, β, and γ-globulins were determined for a series of patient SF samples using aforementioned electrophoresis. Analysis of the SF samples revealed that the total protein concentration was 2-fold greater in the samples than the total protein concentration recommended by ISO-14243-3 (ISO-14243-3 (2004) International Organization for Standardization, London: Implants for Surgery: Wear of Total Knee Joint Prostheses, Part 3: Loading and Displacement Parameters for Wear Testing machines with Displacement Control and Corresponding Environmental Conditions for Test, Displacement Control and Corresponding Environmental Conditions for Test). The mean total protein concentration for SF obtained from osteoarthritic patients was measured to be 34.18±4.78 g/L (range 22.95-43.06 g/L).

The protein constituent fractions in serum lubricants BCS, NCS, ACS, and ACS-I were compared to those concentrations measured for SF (FIG. 4). The protein constituents of the various calf sera were variable both amongst themselves as well as compared to SF. The albumin, α-1-globulin, and α-2-globulin fractions calculated for serum lubricants ACS and ACS-I most closely matched those of SF, compared to protein constituent fractions in either of BCS or NCS. The β-globulin concentration in the SF samples was determined to be similar to those of both NCS and ACS serum lubricants. The γ-globulin fraction of the SF samples was determined to be most similar to those of both BCS and NCS.

### Example 2

### Osmolality and Trace Element Concentrations Vary Depending on the Serum Lubricant and Dilution Medium

Osmolality is a direct measure of the ionic strength of a solution and is a regarded as a systemic, patient specific value (Baumgarten et al. (1985) J. Bone Joint Surg. Am. 67(9):1336-1339). Individual calf sera samples were diluted either with DW or PBS. The choice of dilutive media was suggested to affect solution osmolality given that osmolality has been shown to affect the thermal stability of proteins in solution (Giancola et al. (1997) Int. J. Biol. Macromol. 20(3):193-204) and, thus, might also affect the protein degradation and the PE wear rate in simulator wear testing.

In the present study, an osmometer (Osmometer 5520, Wescor, Logan, Utah) was used to determine the osmolality of the SF, different serum lubricants and their dilutive media. The osmometer determined the osmolality following the freezing-point depression test strategy at atmospheric pressure. This strategy permits the determination of the difference between freezing points of a pure solvent and that of a solution mixed with a solute. The difference between freezing points is directly proportional to the molar concentration of the solution. Prior to testing, the instrument was calibrated using a reference sample that had 290 mmol/kg (Calibration OPTI-Mole™, Wescor, Logan, Utah). Triplicate measurements were obtained for each of the lubricant samples. The osmolality of PBS was 286±0.57 mmol/kg, a value approximately 6-times higher than that of DW (osmolality=46±2.08 mmol·kg). The calf sera were diluted to a final protein concentration of 17 g/L according to ISO 14243-3 using DW or PBS (Cat.#72060-034, VWR, Mississauga, ON) (Table 3).

TABLE 3
Calf serum lubricant diluted with DW or buffer for a final protein
concentration of 17 g/L as recommended by ISO 14243.

| | Dilution | Dilution |
|---|---|---|
| Diluted calf sera | [%] | Media |
| BCS + DW | 25 | DW |
| NCS + DW | 33 | DW |
| ACS + DW | 40 | DW |
| ACS-I + DW | 41.5 | DW |
| BCS + PBS | 25 | PBS |
| NCS + PBS | 33 | PBS |
| ACS + PBS | 40 | PBS |
| ACS-I + PBS | 41.5 | PBS |

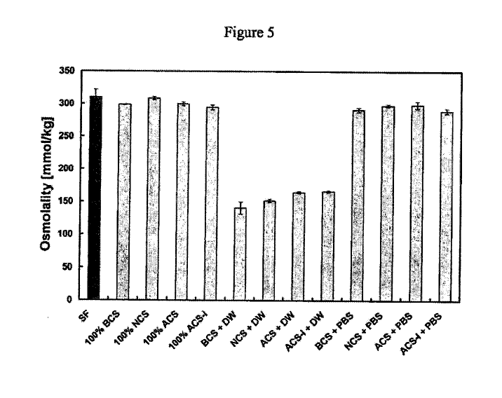

The mean osmolality value for the series of SF samples was measured to be 310.20±11.84 mmol/kg (ranging between 284-324 mmol/kg). The measured mean osmolality value was then compared to osmolality values calculated for various wear simulator testing serum lubricants diluted with either DW or PBS (FIG. 5). The measured osmolalities of undiluted SF, BCS, NCS, ACS, and ACS-I were found to be similar to the osmolality of the respective serum diluted with PBS. Serum dilution with PBS was shown to result in more clinically relevant osmolality levels compared to dilution with DW. The measured osmolality levels were shown to be similar to those of SF.

The concentrations of some trace elements such as calcium (Ca), magnesium (Mg), inorganic phosphorus (P) and iron (Fe) were determined for the SF samples and compared with the concentrations found in the various calf serum mixtures. A chemical analyzer (Synchron LX 20® Pro, Beckman Coulter, Fullerton, Calif.) was utilized to measure these concentrations. Depending on the element, different methodologies were used in the analyzer that is described elsewhere in more detail (Chemistry Information Sheet A18471, Beckman Coulter, Fullerton, Calif., October 2005; Chemistry Information Sheer A18491, Beckman Coulter, Fullerton, Calif., September 2005; Chemistry Information Sheet A18526, Beckman Coulter, Fullerton, Calif., September 2005; Chemistry Information Sheet A18545, Beckman Coulter, Fullerton, Calif., August 2005). Triplicate measurements were obtained for each of the lubricant samples.

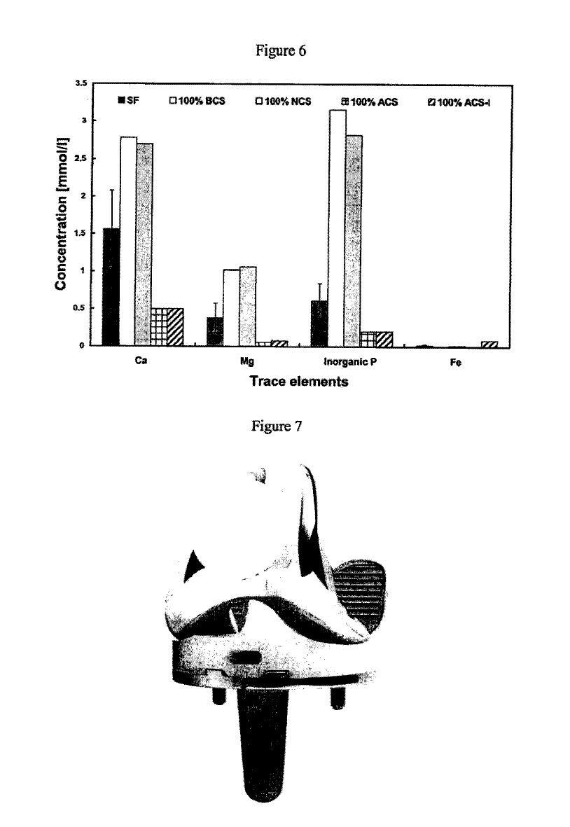

The concentration of Ca, Mg, inorganic P, and Fe were determined in all SF samples, 100% BCS, 100% NCS, 100% ACA, and 100% ACS-I (FIG. 6). The Ca and inorganic P concentration were highest in SF samples followed by Mg and Fe. A similar pattern can be observed for BCS, NCS and ACS, but not for ACS-I with a FE concentration (0.087 mmol/l) close to its Mg concentration (0.080 mmol/l). However, none of the calf sera trace element concentrations were close to the concentrations of human SF samples. While such elements are known to bind or add to proteins and other substances in the SF and calf sera (Stryer (1995) “Biochemistry” (Fourth Edition)), their effect on PE wear remained also unknown.

### Example 3

### Role of Bacterial Growth on Wear Testing

A six million cycles wear tests was performed on the knee simulator (AMTI, Waltham, Mass.) with AMK implant (FIG. 7) components (Table 4) (DePuy Orthopaedics, Warsaw, Ind.). There calf serum lubricants were used: BCS lubricant (BCS+DW+SA), NCS lubricant (NCS+DW+SA), and ACS lubricant (ACS+DW+SA).

TABLE 4
Test protocol from 0-6 Mc.

Lubricant

| Mc | L implants | R implants |
|---|---|---|
| 0-3 | BCS + DW + SA | BCS + DW + SA |
| 3-4.5 | ACS + DW + SA | NCS + DW + SA |
| 4.5-6 | BCS + DW + SA | BCS + DW + SA |

BCS = bovine calf serum;
DW = distilled water;
SA = sodium azide;
ACS = alpha calf serum;
NCS = newborn calf serum.

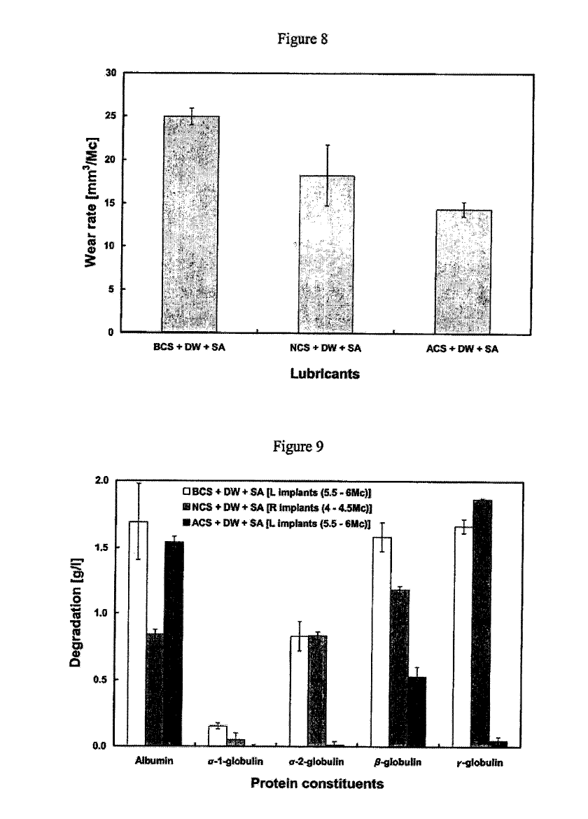

The wear rates obtained from the testing with the BCS lubricant, NCS lubricant, and ACS lubricant indicated that the fractions of serum proteins in the lubricant had a significant effect on wear rate (FIG. 8). The serum lubricants were found to have wear varying rates where BCS lubricant>NCS lubricant>ACS lubricant.

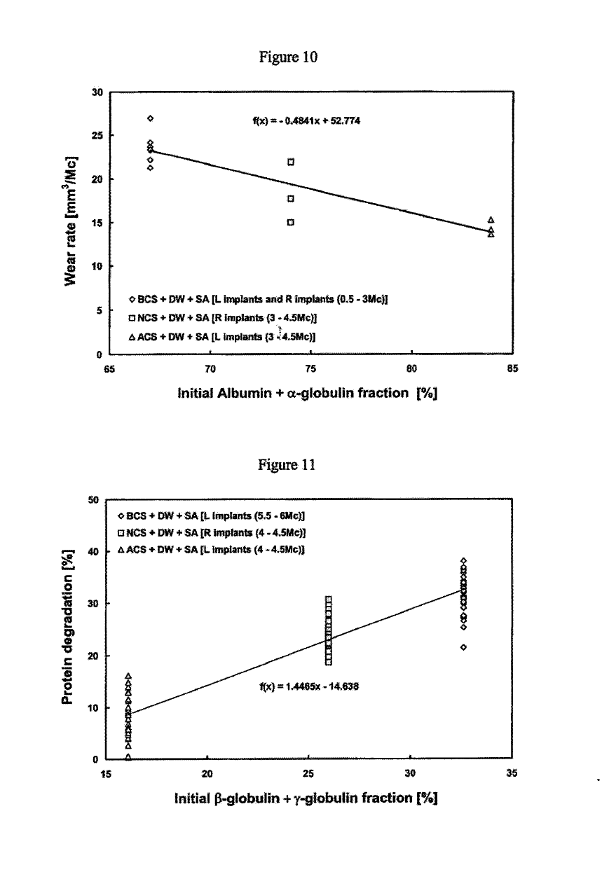

Electrophoresis data obtained for serum lubricant samples suggested that serum protein constituents in the various calf serum lubricants were each affected by the wear process (FIG. 9). Correlation analysis showed that an increased fraction of albumin+α-1-globulin resulted in a reduced PE wear rate (FIG. 10). Elevated β-globulin+γ-globulin fraction correlated with increased protein degradation (FIG. 11).

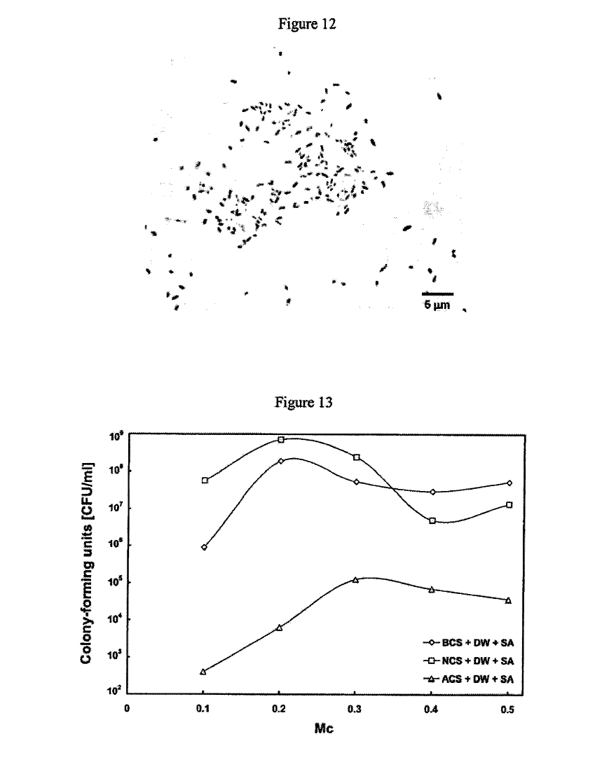

SA was ineffective as a microbial inhibitor in all three lubricants. The microbial contamination was identified as gram-negative E. cloacae, strain JK-1 using the API® 20E system. A micrograph of the organism is shown in FIG. 12.

### Example 4

### Role of Biochemical and Microbiological Effects in Wear Testing

Historically, SA has been widely used to successfully inhibit microbial growth (Lichstein and Soule (1944) J. Bacteriology 47(3): 253-257). However, Bell et al. (2000, Proc. Inst. Mech. Eng. [H] 214(5):513-518) reported microbial growth in pin-on-plate wear tests despite the use of SA-containing lubricant, suggesting that SA is not a useful microbial inhibitor under such conditions. Alternative microbial inhibitors, including specific antibiotic/antimycotics (AA), have been used in wear tests (Schwenke et al. (2005) Proc. Inst. Mech. Eng. [H] 219(6):457-464), however, their effects on wear rates compared to SA have not been established.

Experiments were performed to determine serum protein concentrations in each of starting materials (SM) and supernatants (SUP) in the presence of SA compared to AA. A typical antibiotic/antimycotic composed of penicillin, streptomycin, and amphotericin B (Fungizone®) in saline solution was evaluated for its antimicrobial effects in a wear simulator testing application (Cat. #15240-062, Invitrogen, Missassauga, ON). Implants were evaluated in the presence of different serum lubricants diluted in DW and including one of SA or AA as the microbial inhibitor. AA was added at a concentration of 1% per lubricant volume at the beginning of the wear tests and was again repeatedly added every ˜0.16 Mc of wear testing to uphold the efficacy of the microbial agent.

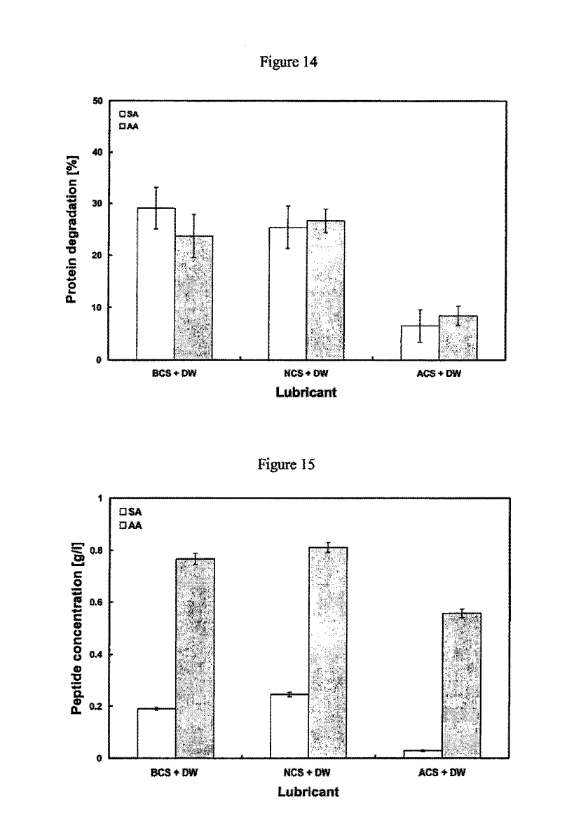

Lubricants from the wear stations were sampled at the start of the tests (0 Mc) and every 0.1 Mc after that and the lubricant samples were then plated in triplicate on LB agar. No bacterial growth was detected in any of the wear stations at 0 Mc. However, bacterial growth was observed after the first 0.1 Mc in all the wear stations where SA was used as the microbial inhibitor (FIG. 13). NCS+DW+SA appeared to exhibit higher levels of bacterial growth compared to BCS+DW+SA up to 0.35Mc. In general, the bacterial growth was reduced in tests using ACS, compared to other serums, as the lubricant. Bacterial growth was completely inhibited in wear simulator tests using AA as microbial inhibitor in the case of consecutive tests of 0.5 Mc using BCS+DW, NCS+DW, and ACS+DW lubricants. Lubricant serum protein degradation was evident following wear testing regardless of the used microbial agent (FIG. 14). The peptide concentration was significantly higher for in all calf sera lubricants when SA was replaced with AA (FIG. 15).

### Example 5

### Effects of Antimicrobial Agents on Protein Degradation

It was previously shown that the thermal stability of the lubricant affected the tribology at the CoCr-PE interface (1-leuberger et al. (2005) Biomaterials 26(10):1165-1173; Fang et al. (2007) Appl. Surface Sci. 253(16):6896-6904). In order to obtain information concerning the thermal stability (transition midpoint temperature Tₘ; enthalpy change ΔH; entropy change, ΔS) of the lubricants, triplicate samples of various ACS-I lubricants as well as SF were subjected to Differential Scanning calorimetry (DSC).

The protein stability of a dilute protein solution depends on the partial molar heat capacity at constant pressure, cₚ. The change in cₚ reflects the ability of the protein solution to absorb heat and cope with a defined increase in temperature. A protein in a dilute solution is in equilibrium between the native (folded) conformation and its denatured (unfolded) conformation.

The stability of the native state is based on the magnitude of the Gibbs free energy (ΔG) of the system and the thermodynamic relationships between enthalpy (ΔH) and entropy (ΔS) changes: ΔG=ΔH−TΔS. A positive ΔG indicates the native state is more stable than the denatured state; the more positive the ΔG, the greater the stability.

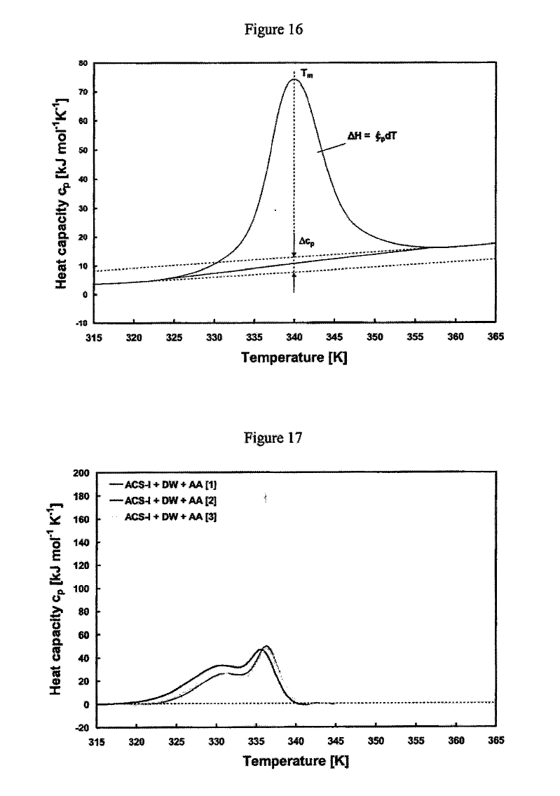

In order to allow a protein to become unfolded, stabilizing forces need to be broken. Protein unfolding occurs when the entropic changes are significantly increased to overcome stabilizing enthalpic interactions between protein hydrogen bonds, hydrophobic interactions and electrostatic interactions, resulting in an endothermic peak at a certain transition midpoint temperature Tₘ (FIG. 16). The Tₘ directly indicates the thermal stability of the protein. The higher the Tₘ the more stable is the protein at lower temperatures. ΔH is primarily due to changes in hydration of side chains that are buried in the native state and become exposed in the denatured state. The shift in baseline before and after the transition represents the change in Δcₚ of the protein in association with the solvent caused by unfolding.

The sharpness of the transition peak is an index of the cooperative nature of the transition from native formation to denatured formation. The unfolding process becomes a multi-stage process when more than one peak is observed which means it is less cooperative. At temperature below Tₘ the concentration of native proteins is higher than of denatured. The Tₘ at maximal cₚ (T_{m(cp-max)} is referred to as the transition point where half the proteins are folded and half are unfolded. At this point ΔG=0 and the conformational entropy ΔS=ΔH/T_{m(cp-max)} can be directly calculated. During unfolding, a protein transforms from a single folded confirmation to many random unfolded conformations. ΔS is the measure of disorder/randomness in a system and an increase in ΔS indicates the amount energy dissipated to transform proteins from native, folded conformation to random, unfolded conformation. Higher ΔS indicates higher conformational protein stability. Conformational entropy overcomes the stabilizing forces allowing the protein to unfold at temperatures where entropy becomes dominant.

A MicroCal VP-DSC calorimeter (MicroCal, Northhampton, Mass.) was used in the present study to obtain the thermogram for several lubricant mixtures. Firstly, a baseline for a thermogram was established by scanning a buffer solution which consists of the media used to dilute the calf serum. Secondly, each lubricant was diluted with their according buffer solution to a total protein concentration of 6 g/l and were subsequently scanned. The scan rate was 60 K/hour was applied to 283 K to 368 K. The concentration molality was uniformly set at 0.05 mmol/L. A relative comparison of Tₘ, ΔH and ΔS was established. The contribution of the serum proteins on the calorimetrically measured cₚ was determined by subtracting a scan of the buffer solution from the data prior to lubricant analysis. Such a procedure ensured that any effects of the solvent on the proteins were eliminated and the thermal signal is entirely due to the serum constituents. A software program (Origin 5.0, MicroCal, Northhampton, Mass.) was used to analyze the data. Tₘ, ΔH, and ΔS of the transition were calculated by fitting the data to a two-state transition model using non-linear least squares regression analysis (Levenberg-Marquardt non-linear least-square method) (More (1977) “Levenberg-Marquardt algorithm: implementation and theory, United States, pp. 13). The unfolding of the protein constituents of the calf serum based lubricants was considered an irreversible process (Pico (1997) Int. J. Biological Macromolecules 20(1):63-73).

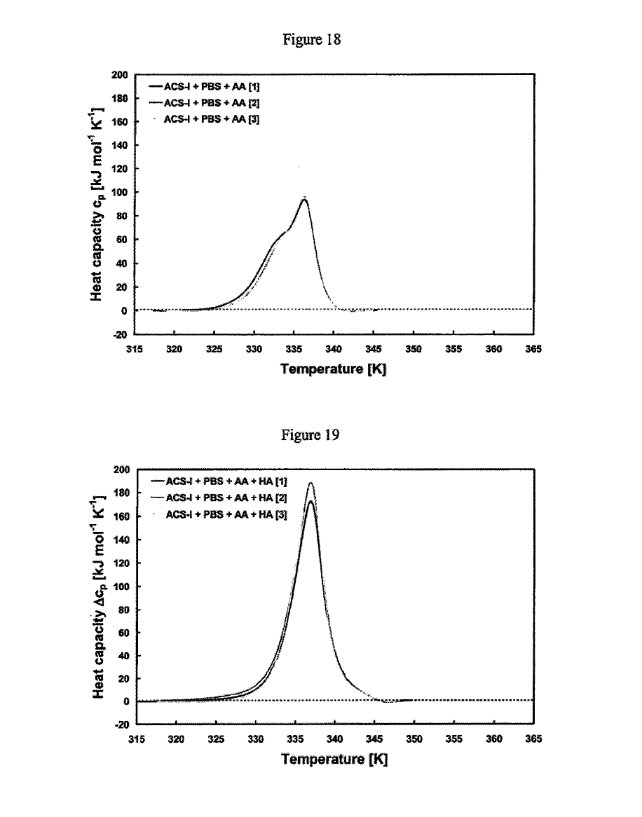

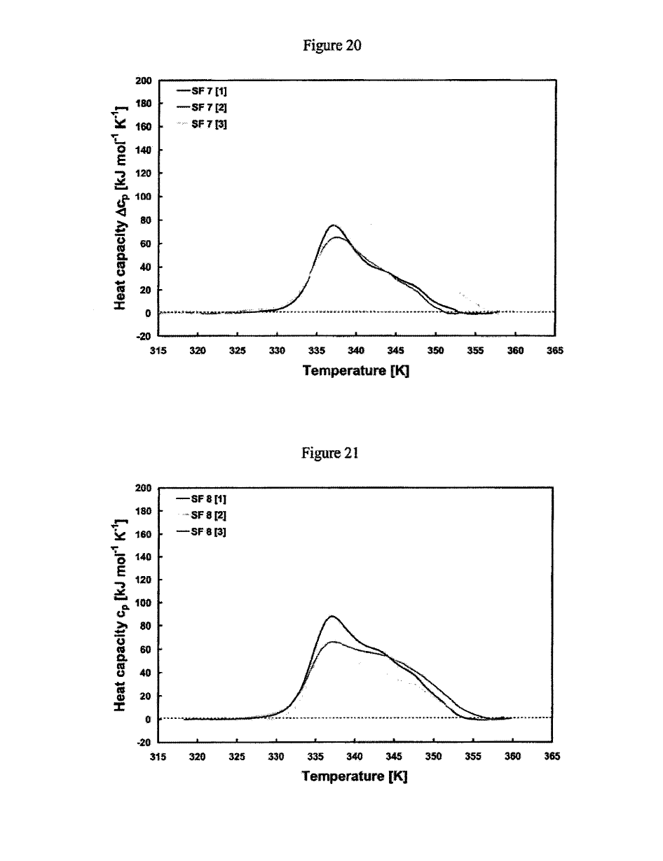

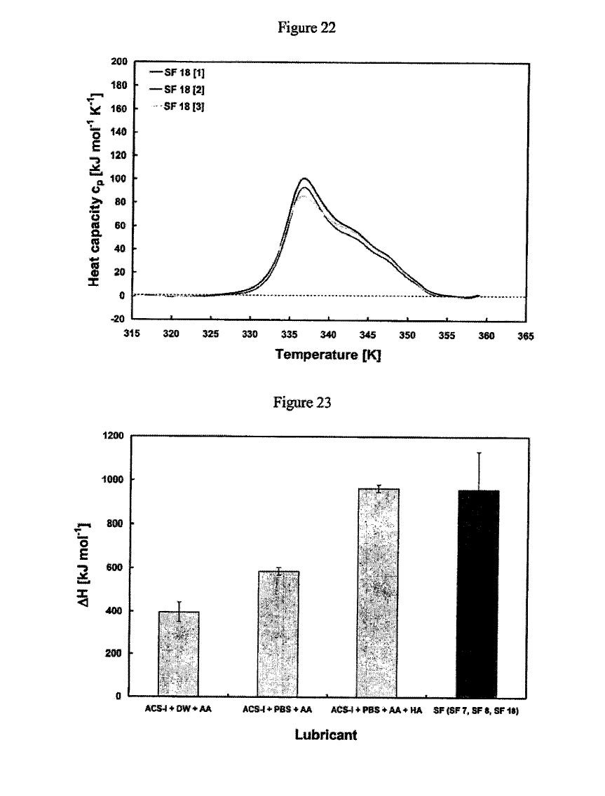

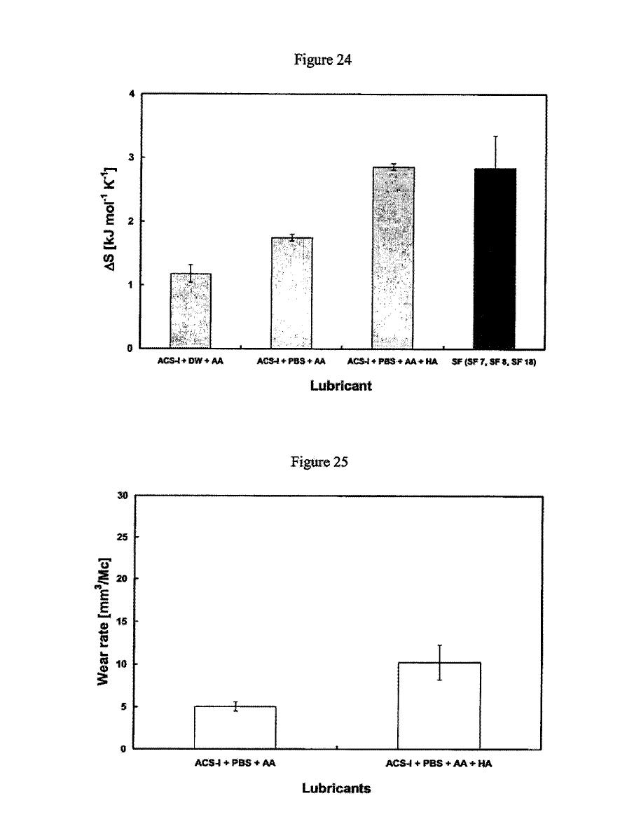

To gain some insight into the thermal stability of the lubricants triplicate samples of the DW lubricant (ACS-I+DW+AA), the PBS lubricant (ACS-I+PBS+AA), the HA lubricant (ACS-I+PBS+AA+HA) and SF (samples of SF 7, SF 8, and SF 18) were analyzed using Differential Scanning calorimetry (DSC) methods. The buffer solutions in the DSC tests were DW+AA, PBS+AA, PBS+AA+HA and PBS for the lubricants ACS-I+DW+AA, ACS-I+PBS+AA, ACS-I+PBS+AA+HA, and SF, respectively. DW lubricant and ACS-I+PBS+AA had a significantly lower enthalpy change (ΔH) entropy change (ΔS) compared with ACS-I+PBS+AA+HA and SF (FIGS. 17-24). Both the transition midpoint temperature (Tₘ₁) and the maximal specific heat at constant pressure (cₚ) increased when ACS-I+AA was diluted with PBS instead with DW. Such change in dilutive media increased the osmolality of the lubricant from 145±2.00 mmol/kg to 321±2.64 mmol/kg. However, Tₘ₁ for PBS lubricant was lower compared with SF. The DW lubricant and PBS lubricant did not have a Tₘ₃. The unfolding of the HA lubricant was highly cooperative (only one very well defined cₚ-peak during unfolding) and had a comparable Tₘ₁ to SF. The unfolding of SF samples was a multi-stage process showing three Tₘ, with Tₘ₁ having the highest cₚ (Table 5). The unfolding of SF was consequently less cooperative (several cₚ-peaks during the unfolding) than for the HA lubricant. ACS-I+DW+AA had a lower Tₘ₁ and T_{m(cp-max)} compared with SF. The thermograms of SF were similar between samples SF 7, SF 8, SF 18. The unfolding of SF was less cooperative than ACS-I+PBS+AA+HA but showed similar in T_{m(cp-max)}, ΔH and the change in entropy (ΔS). The ΔS for the lubricants was almost proportional to ΔH since the T_{m(cp-max)} was of the same order of magnitude between the lubricants.

TABLE 5
Transition midpoint temperatures, Tₘ, of the DW lubricant,
PBS lubricant, HA lubricant, SF 7, SF 8, and SF 18 (n = 3).

| | Transition midpoint | | |
|---|---|---|---|
| | temperature Tₘ [K] | | |
| Lubricant | Tₘ₁ | Tₘ₂ | Tₘ₃ |
| ACS-I + DW + AA | 330.71 ± 1.55 | 336.30 ± 1.22 | N/A |
| ACS-I + PBS + AA | 333.39 ± 1.05 | 336.46 ± 0.05 | N/A |
| ACS-I + PBS + AA + | 337.32 ± 0.58 | N/A | N/A |
| HA | | | |
| SF 7 | 337.32 ± 1.83 | 342.87 ± 1.33 | 347.72 ± 3.87 |
| SF 8 | 336.73 ± 0.52 | 342.63 ± 1.73 | 348.40 ± 2.22 |
| SF 18 | 336.35 ± 2.13 | 341.26 ± 4.26 | 347.48 ± 2.01 |

### Example 6

### The Effects of Lubricant Osmolality and Hyaluronic Acid on PE Wear

The types of calf serum based lubricants used for implant wear testing vary between independent research laboratories and implant manufacturers (Clarke et al. (2001) Wear 250(1-12):188-198). Wang et al (2004, J. Biomed. Mater. Res. 68B(1):45-52) suggested that the test lubricant should be classified based on physiological A/G ratio. They also added 0.34 g/l HA to the lubricant with SA as the microbial inhibitor and did not observe an effect on the PE wear rate in their hip simulator study. In contrast, Desjardins et al. (2006, Proc. Inst. Mech. Eng. [H] 220(5):609-623) added 1.5 g/L HA having a molecular weight of 2.3 MDa to bovine calf serum, and incorporated SA as the microbial inhibitor, and observed a 6.8-fold in increase in PE wear rate. Mazzucco et al. (2003, “Variation in Joing Fluid Composition and its Effect on the Tribology of Replacement Joint Articulation”, Ph.D. thesis, MIT) reported a mean molecular weight for HA of 1.8 MDa, at a mean concentration of 1.5 g/L in SF.

A 5.5 Mc wear test was performed on the aforementioned implant components, the results of which are provided in Table 6. The average PE wear rate for the L implants increased 2-times from 5.04±0.56 mm³/Mc to 10.24±2.04 mm³/Mc when HA was added to the lubricant (FIG. 25). Such a change was statistically significant (p=0.013, Student's t-test).

TABLE 6
Test protocol showing the lubricant compositions and
their corresponding osmolality levels.

| | Lubricants | |
|---|---|---|
| Test interval | (Osmolality [mmol/kg]) | |
| [Mc] | L implants (L1, L2, L3) | R implants (R1, R2, R3) |
| 0-3.5 | ACS-I + PBS + AA | ACS-I + PBS + AA |
| | (312 ± 1.00) | (312 ± 1.00) |
| 3.5-5.5 | ACS-I + PBS + AA + HA | ACS-I + DW + AA |
| | (321 ± 2.64) | (145 ± 2.00) |

ACS-I + PBS + AA is referred to as the “PBS lubricant”;
ACS-I + DW + AA is referred to as the “DW lubricant”, and
ACS-I + PBS + AA + HA is referred to as the “HA lubricant”.

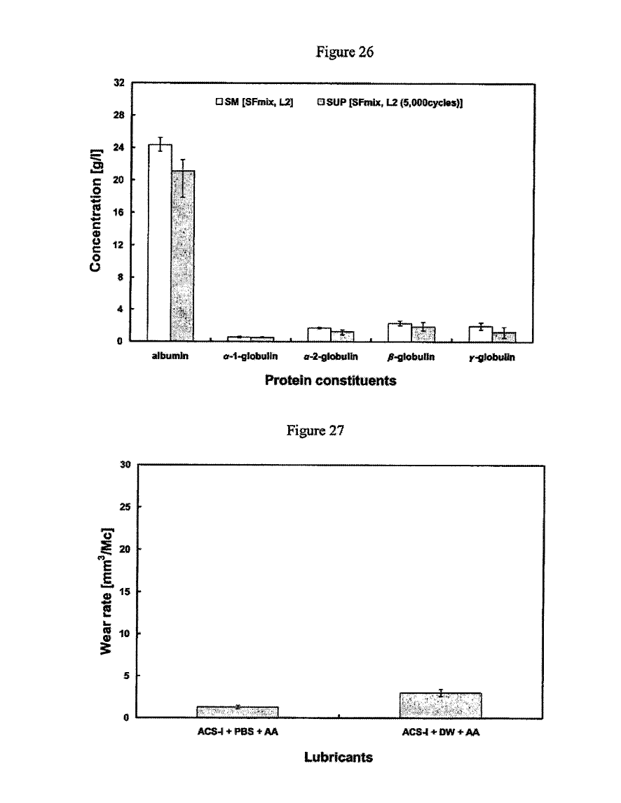

A 5,000 cycle (83 min) “SF wear test” with 10 ml of mixed SF was performed on implant L2. SF samples of 10 patients (SF 3, SF 4, SF 5, SF 6, SF 7, SF 8, SF 11, SF 13, SF 14, and SF 16) were mixed (SFₘᵢₓ). The SFₘᵢₓ had a protein concentration of 30.90±1.24 g/l and a peptide concentration of 0.397±0.004 g/l. The test was performed in an open system and the SFₘᵢₓ was repeatedly pipetted (every 3-5 cycles) onto the anterior aspect of the femoral component which allowed the SFₘᵢₓ to be dragged into the interface by the articulating surfaces. The protein concentration of the SUP measured 25.98±1.31 g/l. The peptide concentration in SFₘᵢₓ increased by 56% to 0.618±0.010 g/l. The electrophoretic profile of the SFₘᵢₓ was different between the SM and the SUP (FIG. 26).

The mean PE wear rate for the R implants increased 2.3-times from 1.29±0.17 mm³/Mc to 2.99±0.16 mm³/Mc when PBS was replaced with DW as the dilutive media. This change appeared statistically significant (p=0.003, Student's t-test). Using DW as the dilutive media for ACS-I resulted in non-clinically relevant osmolality levels and increased the PE wear rate. Using PBS as the dilutive media reproduced clinically relevant osmolality levels and increased the thermal stability and reduced the wear rate (FIG. 27).

### Example 7

### Effects of Antimicrobial Agents on Peptide Concentration

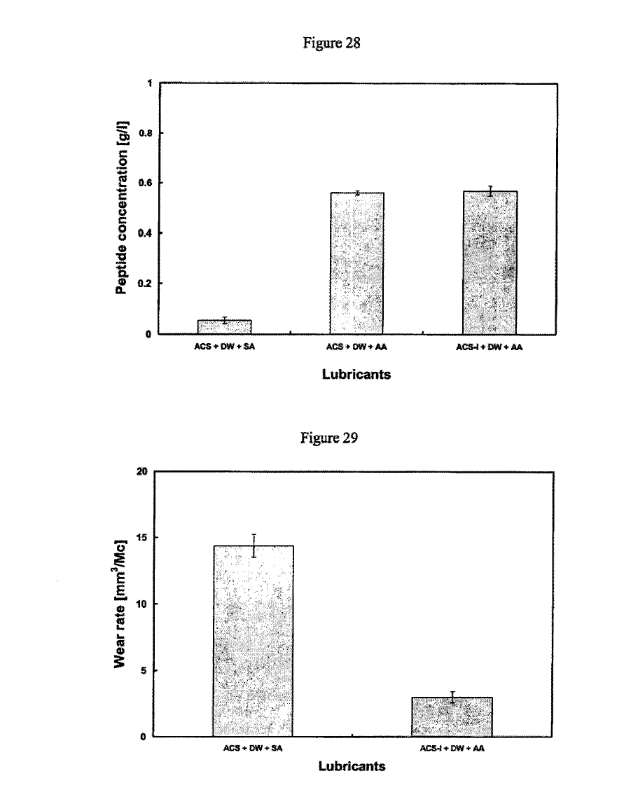

The wear rate was significantly different between the L bank and the R bank when ACS-I+PBS+AA was used as lubricant (FIGS. 25 and 27). The simulator was considered to be of two banks that are independent of each other. The data showed that the wear rates of the lubricants were higher when SA was used as the microbial inhibitor compared with AA (FIGS. 8, 25, and FIG. 27). Microbial growth in the lubricants was associated with reduced peptide concentration, suggesting that the E. cloacae JK-1 metabolized such peptides. Comparison of the peptide concentrations in lubricants ACS+DW+SA, ACS+DW+AA, and ACS-I+DW+AA showed that the peptide concentration was increased 11-fold when SA was replaced with AA (FIG. 28).

The protein constituent concentration between ACS and ACS-I were found to be similar (Table 2). There did not appear to be a significant difference between the peptide concentrations when iron-rich ACS-I was used (p=0.170, ANOVA and Tamhane) and thus, the effects of Fe on peptide concentration appeared to be low. EDTA was then added to all lubricants, which should guarantee the binding of free Fe. Thus, it may be reasonable to compare the wear rates ACS+DW+SA with the war rate of ACS-I+DW+AA; replacing SA with AA caused a 4-times decrease in PE wear rate (FIG. 29).

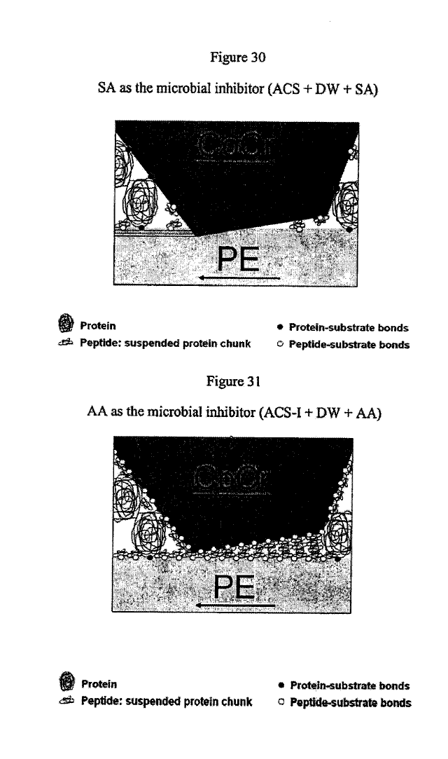

The peptides in the lubricant appeared to form a thin peptide layer (conditioning film), with an average molecular weight of 2,000 Da (FIGS. 30, 31). This protein layer was suggested to cover the bearing surface with more low molecular weight brushes than a lubricant with a low peptide concentration, requiring more energy to be squeezed out of the contact zone to onset adhesive/abrasive wear (Persson and Mugele (2004) J. Physics: Condensed Matter 16(10):R295-R355).

All of the methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and in the steps or sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

1. An artificial synovial fluid composition comprising:
a) a mammalian serum selected from the group consisting of alpha calf serum (ACS) and iron-supplemented alpha calf serum (ACS-I) having:
i) a total protein concentration of from about 10 to about 45 g/L;
ii) an albumin fraction of from about 55% to about 80% w/w %;
iii) an α-1 globulin fraction of from about 3% to about 6% w/w %;
iv) an α-2 globulin fraction of from about 5% to about 10% w/w %;
v) a β-globulin fraction of from about 3% to about 20% w/w %;
vi) a γ-globulin fraction of from about 5% to about 20% w/w %;
vii) a calcium (Ca) concentration of from about 0.1 mmol/L to about 3 mmol/L;
viii) a magnesium (Mg) concentration of from about 0.05 mmol/L to about 0.8 mmol/L;
ix) an inorganic phosphate (P) concentration of from about 0.1 mmol/L to about 1.5 mmol/L;
x) an iron (Fe) concentration of from about 0.001 mmol/L to about 0.1 mmol/L;
xi) a peptide concentration at a 2,000 Da molecular weight cut-off of from about 0.005 g/L to about 10 g/L;
xii) a maximal transition midpoint temperature, T_{m-cp-ma}, of from about 330K to about 350K at a concentration molality of from about 0.005 to about 0.15 mmol/L measured at a concentration molality of from about 0.005 mmol/l to about 0.15 mmol/l and a scan rate from about 20K/hour to 100K/hour;
xiii) a total enthalpy change, ΔH, of from about 650 kJ mol⁻¹ to about 1200 kJ mol⁻¹ at a concentration molality of from about 0.005 mmol/l to about 0.15 mmol/l and a scan rate from about 20K/hour to 100K/hour;
xiv) a total entropy change, ΔS, of from about 1.5 kJ mol⁻¹ K⁻¹ to about 4 kJ mol⁻¹ K⁻¹ at a concentration molality of from about 0.005 mmol/l to about 0.15 mmol/l and a scan rate from about 20K/hour to 100K/hour;
wherein the ratio of albumin to total globulin (α-1 globulin+α-2 globulin+β-globulin+γ-globulin fractions) in solution is from about 1.5 to about 3; and
b) a buffer for maintaining a solution osmolality of from about 200 mmol/kg to about 400 mmol/kg;
c) hyaluronic acid (HA) at a concentration of from about 0.1 g/L to about 6 g/L; and
d) hyaluronic acid (HA) with a molecular weight of from 1 MDa to 4 MDa; and
e) a pH level of about 7 to about 8;
wherein the artificial synovial fluid composition mimics the tribological properties of human, osteoarthritic synovial fluid.

2. The artificial synovial fluid of claim 1, further comprising a microbial inhibitor selected from the group consisting of a β-lactam antibiotic; a β-lactamase-sensitive penicillin; a β-lactamase inhibitor; a combination β-lactam antibiotic and beta-lactamase inhibitor; an aminoglycoside; and a bacteriostatic, wherein the microbial inhibitor is present at a concentration of from about 1% to about 15% total volume.

3. The artificial synovial fluid of claim 1 further comprising an antifungal agent.

4. The artificial synovial fluid of claim 3, wherein the antifungal agent is selected from the group comprising Amphotericin B, Fungilin, Abelcet, AmBisome, Fungisome, Amphocil, and Amphotec.

5. The artificial synovial fluid of claim 2, wherein the β-lactam antibiotic is selected from the group comprising Ampicillin, Pivampicillin, Carbenicillin, Amoxicillin, Carindacillin, Bacampicillin, Epicillin, Pivmecillinam, Azlocillin, Mezlocillin, Mecillinam, Piperacillin, Ticarcillin, Metampicillin, Talampicillin, Sulbenicillin, Temocillin, and Hetacillin.

6. The artificial synovial fluid of claim 2, wherein the β-lactamase sensitive penicillin is selected from the group comprising Benzylpenicillin, Phenoxymethylpenicillin, Propicillin, Azidocillin, Pheneticillin, Penamecillin, Clometocillin, Benzathine benzylpenicillin, Procaine benzylpenicillin, Benzathine phenoxymethylpenicillin, Dicloxacillin, Cloxacillin, Methicillin, Oxacillin, and Flucloxacillin.

7. The artificial synovial fluid of claim 2, wherein the β-lactamase inhibitor is selected from the group comprising Sulbactam and Tazobactam.

8. The artificial synovial fluid of claim 2, wherein the combination penicillin and beta-lactamase inhibitor is selected from the group comprising Ampicillin and an enzyme inhibitor; Amoxicillin and an enzyme inhibitor, Ticarcillin and enzyme inhibitor, Sultamicillin Piperacillin and enzyme inhibitor, combinations of penicillins including beta-lactamase inhibitors (Ampicillin and enzyme inhibitor, Amoxicillin and enzyme inhibitor, Ticarcillin and enzyme inhibitor, Sultamicillin Piperacillin and enzyme inhibitor, combinations of penicillins).

9. The artificial synovial fluid of claim 2, wherein the aminoglycoside is selected from the group comprising Streptomycin, Streptoduocin, Tobramycin, Gentamicin, Kanamycin, Neomycin, Amikacin, Netilmicin, Sisomicin, Dibekacin, Ribostamycin, and Isepamicin.

10. The artificial synovial fluid of claim 2, wherein the bacteriostatic is selected from the group comprising Tetracycline, Sulphonamide, Spectinomycin, Trimethoprim, Chloramphenicol, Macrolide and Lincosamide.

11. The artificial synovial fluid of claim 1, wherein the mammalian serum is a bovine serum selected from the group comprising alpha calf serum (ACS), ACS-I, bovine calf serum (BCS) and newborn calf serum (NCS).

12. The artificial synovial fluid of claim 1, wherein the buffer comprises sodium chloride, sodium phosphate and potassium phosphate.

13. The artificial synovial fluid composition of claim 12, wherein the buffer is PBS.

14. The artificial synovial fluid composition of claim 1, wherein the total protein concentration is from about 22 g/L to 44 g/L.

15. The artificial synovial fluid composition of claim 1, wherein the albumin fraction is from about 60% to about 75%.

16. The artificial synovial fluid composition of claim 1, wherein the α-1 globulin fraction is from about 3.3% to about 5.2%.

17. The artificial synovial fluid composition of claim 1, wherein the α-2 globulin fraction is from about 5.8% to about 9.7%.

18. The artificial synovial fluid composition of claim 1, wherein the β-globulin fraction is from about 3.4% to about 17.7%.

19. The artificial synovial fluid composition of claim 1, wherein the γ-globulin fraction is from about 4.8% to about 12.9%.

20. The artificial synovial fluid composition of claim 1, wherein the hyaluronic acid (HA) concentration is from about 1 g/L to about 2 g/L.

21. The artificial synovial fluid composition of claim 1, wherein the molecular weight of hyaluronic acid (HA) is from about 1.5 MDa to about 3.5 MDa.

22. The artificial synovial fluid composition of claim 1, wherein the pH level is from about 7.1 to about 7.8.

23. The artificial synovial fluid composition of claim 1, wherein the calcium (Ca) concentration is from about 0.32 mmol/L to about 2.2 mmol/L.

24. The artificial synovial fluid composition of claim 1, wherein the magnesium (Mg) concentration is from about 0.04 mmol/L to about 0.7 mmol/L.

25. The artificial synovial fluid composition of claim 1, wherein the inorganic phosphate (P) concentration is from about 0.2 mmol/L to about 1.1 mmol/L.

26. The artificial synovial fluid composition of claim 1, wherein the iron (Fe) concentration is from about 0.004 mmol/L to about 0.05 mmol/L.

27. The artificial synovial fluid composition of claim 1, wherein the peptide concentration at a 2,000 Da molecular weight cut-off is from about 0.05 g/L to about 2 g/L.

28. The artificial synovial fluid composition of claim 1, wherein the maximal transition midpoint temperature, T_{m-cp-max}, is from about 336 K to about 338 K at a concentration molality of from about 0.005 mmol/l to about 0.15 mmol/l and a scan rate from about 20K/hour to 100K/hour.

29. The artificial synovial fluid composition of claim 1, wherein the total enthalpy change, ΔH, is from about 700 kJ mol⁻¹ to about 1050 kJ mol⁻¹ at a concentration molality of from about 0.005 mmol/l to about 0.15 mmol/l and a scan rate from about 20K/hour to 100K/hour.

30. The artificial synovial fluid composition of claim 1, wherein a total entropy change, ΔS, is from about 2 kJ mol⁻¹ K⁻¹ to about 3.2 kJ mol⁻¹ K⁻¹ at a concentration molality of from about 0.005 mmol/l to about 0.15 mmol/l and a scan rate from about 20K/hour to 100K/hour.

31. The artificial synovial fluid composition of claim 1, wherein the ratio of albumin to total globulin (α-1 globulin+α-2 globulin+β-globulin+γ-globulin fractions) in solution is from about 1.8 to about 2.3.

32. A method of joint implant wear simulator testing in vitro comprising subjecting the joint implant to wear simulator testing in the presence of an artificial synovial fluid composition that mimics the tribological properties of human, osteoarthritic synovial fluid as claimed in claim 1.

33. The artificial synovial fluid composition of claim 1, wherein the solution osmolality is from about 284 mmol/kg to about 334 mmol/kg.

34. An artificial synovial fluid composition comprising:
a) a mammalian serum having:
i) a total protein concentration of from about 10 to about 45 g/L;
ii) an albumin fraction of from about 55% to about 80% w/w %;
iii) an α-1 globulin fraction of from about 3% to about 6% w/w %;
iv) an α-2 globulin fraction of from about 5% to about 10% w/w %;
v) a β-globulin fraction of from about 3% to about 20% w/w %;
vi) a γ-globulin fraction of from about 5% to about 20% w/w %;
vii) a calcium (Ca) concentration of from about 0.1 mmol/L to about 3 mmol/L;
viii) a magnesium (Mg) concentration of from about 0.05 mmol/L to about 0.8 mmol/L;
ix) an inorganic phosphate (P) concentration of from about 0.1 mmol/L to about 1.5 mmol/L;
x) an iron (Fe) concentration of from about 0.001 mmol/L to about 0.1 mmol/L;
xi) a peptide concentration at a 2,000 Da molecular weight cut-off of from about 0.005 g/L to about 10 g/L;
xii) a maximal transition midpoint temperature, T_{m-cp-max}, of from about 330K to about 350K at a concentration molality of from about 0.005 to about 0.15 mmol/L measured at a concentration molality of from about 0.005 mmol/l to about 0.15 mmol/l and a scan rate from about 20K/hour to 100K/hour;
xiii) a total enthalpy change, ΔH, of from about 650 kJ mol⁻¹ to about 1200 kJ mol⁻¹ at a concentration molality of from about 0.005 mmol/l to about 0.15 mmol/l and a scan rate from about 20K/hour to 100K/hour.
xiv) a total entropy change, ΔS, of from about 1.5 kJ mol⁻¹ K⁻¹ to about 4 kJ mol⁻¹ K⁻¹ at a concentration molality of from about 0.005 mmol/l to about 0.15 mmol/l and a scan rate from about 20K/hour to 100K/hour.
wherein the ratio of albumin to total globulin (α-1 globulin+α-2 globulin+β-globulin+γ-globulin fractions) in solution is from about 1.5 to about 3; and
b) a buffer for maintaining a solution osmolality of from about 284 mmol/kg to about 334 mmol/kg;
c) hyaluronic acid (HA) at a concentration of from about 0.1 g/L to about 6 g/L; and
d) hyaluronic acid (HA) with a molecular weight of from 1 MDa to 4 MDa; and
e) a pH level of about 7 to about 8
wherein the artificial synovial fluid composition mimics the tribological properties of human, osteoarthritic synovial fluid.
